(19) United States
(12) Patent Application Publication
(10) Pub. No.: US 2026/0199299 A1
(43) Pub. Date: Jul. 16, 2026

(54) BETA CARBOLINES AS TOPICAL ANTI-INFLAMMATORY AGENTS FOR APPLICATION TO MUCOUS MEMBRANES

(71) Applicant: President and Fellows of Harvard College, Cambridge, MA (US)

(72) Inventors: Smita Gopinath, Cambridge (MA); Morgan Martin, Cambridge (MA); Jon C. Clardy, Cambridge (MA); Ki Hyun Kim, Cambridge, MA (US); Sunghee Bang, Cambridge (MA)

(21) Appl. No.: 18/872,479

(22) PCT Filed: Jun. 8, 2023
(86) PCT No.: PCT/US2023/024801

§ 371 Date: Dec. 6, 2024

Related U.S. Application Data
(60) Provisional application No. 63/350,169, filed on Jun. 8, 2022.

(51) Int. Cl. A61K 31/437; A61P 37/06; C07D 471/04

(52) U.S. Cl. CPC A61K 31/437; A61P 37/06; C07D 471/04

(57) ABSTRACT

Described herein are vaginal lactobacilli strains that actively modulate mucosal immunity, and compounds produced by these bacteria that exhibit anti-inflammatory activity.

### RELATED APPLICATIONS

This application claims the benefit of U.S. Provisional Patent Application No. 63/350,169, filed on Jun. 8, 2022. The contents of this application are hereby incorporated by reference in their entirety.

### STATEMENT REGARDING FEDERALLY SPONSORED RESEARCH

This invention was made with government support under Grant Numbers AI142720 and AI049928 awarded by the National Institutes of Health. The government has certain rights in the invention.

### BACKGROUND

Resident bacteria that live on our mucosal surfaces secrete compounds that can affect our host immune response. While most microbiome research has been focused on the intestinal tract, the vaginal microbiome remains largely overlooked. Unlike the gut microbiome, the vaginal microbiome is composed of 3-5 bacterial communities, the majority of which are dominated by a single Lactobacillus species. Loss of lactobacilli dominance is strongly correlated with multiple adverse health outcomes including increased susceptibility to sexually transmitted infections, increased vaginal inflammation characterized by secretion of pro-inflammatory cytokines, and increased risk of pre-term birth.

To address the adverse health outcomes associated with loss of lactobacilli dominance, restoring lactobacilli dominance to the vaginal microbiome has been proposed; however, evidence for sustained lactobacilli colonization has been lacking. A recent randomized trial demonstrated that only half of the participants retained the probiotic strain.

There exists a need for topical anti-inflammatory agents for use in the vagina and on other mucosal surfaces for the prevention and treatment of the adverse health outcomes associated with loss of lactobacilli dominance.

### SUMMARY

In certain aspects, the present disclosure provides methods of treating or preventing inflammation or treating or preventing a viral infection, comprising administering to a subject in need thereof a therapeutically effective amount of a compound of formula (Ia):

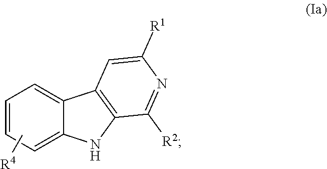

or a pharmaceutically acceptable salt thereof,
wherein
   R¹ is selected from H,

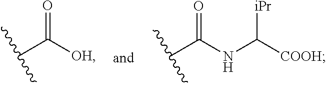

R² is selected from

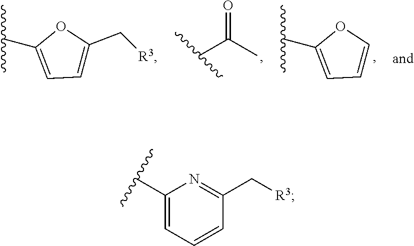

R³ is selected from OH, OCH₃,

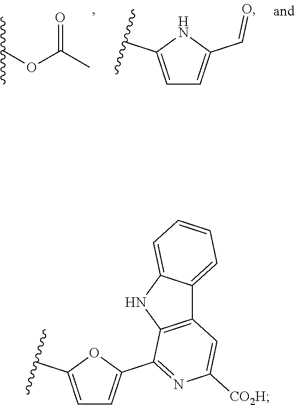

and
R⁴ is selected from H, halo, and C₁-C₄ alkyl.

In certain aspects, the present disclosure provides methods of treating or preventing inflammation or treating or preventing a viral infection, comprising administering to a subject in need thereof a therapeutically effective amount of a compound of formula (I):

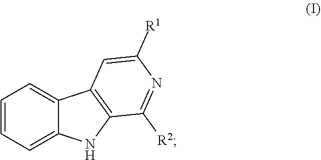

or a pharmaceutically acceptable salt thereof,
wherein
   R¹ is selected from H,

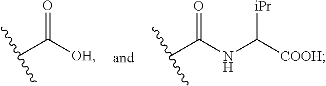

R² is selected from

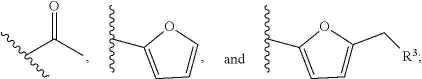

and
R³ is selected from OH, OCH₃,

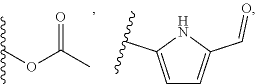

and

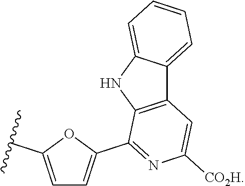 [SMILES: *c1ccc(-c2nc(C(=O)O)cc3c2[nH]c2ccccc23)o1]

In other aspects, the disclosure describes methods of treating or preventing inflammation or treating or preventing a viral infection, comprising administering to a subject in need thereof a therapeutically effective amount of a compound of formula (II):

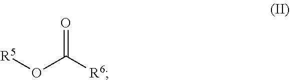

or a pharmaceutically acceptable salt thereof,
wherein
   R⁵ is selected from H

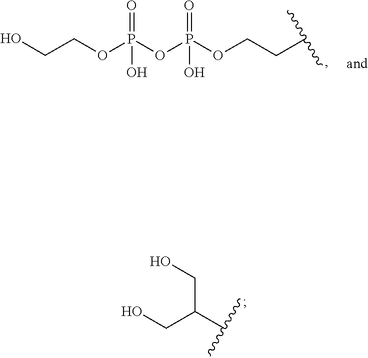 [SMILES: *CCOP(=O)(O)OP(=O)(O)OCCO ; *C(CO)CO]

and
R⁶ is optionally substituted C₈-C₂₆ alkyl.

In certain embodiments, the disclosure provides a compound selected from:

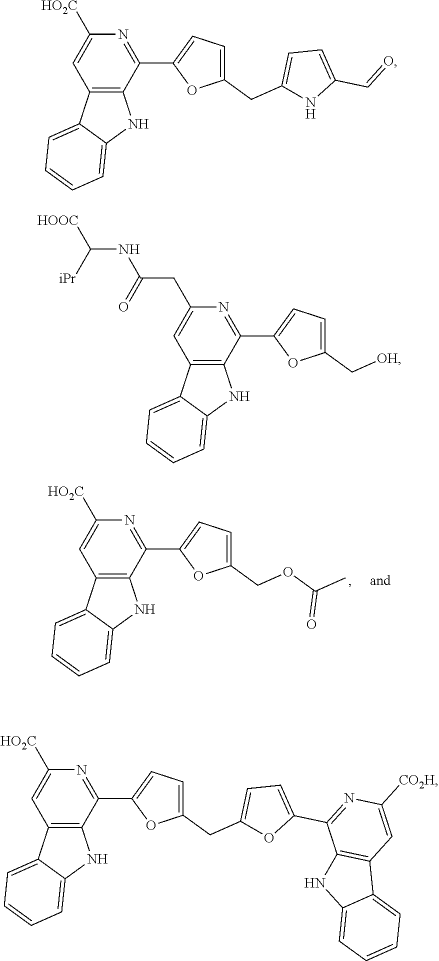

or a pharmaceutically acceptable salt thereof.

In certain embodiments, the disclosure provides a compound selected from

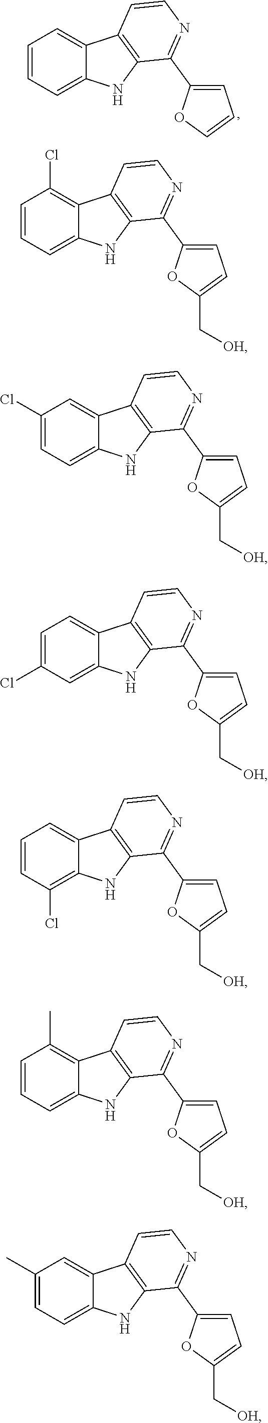

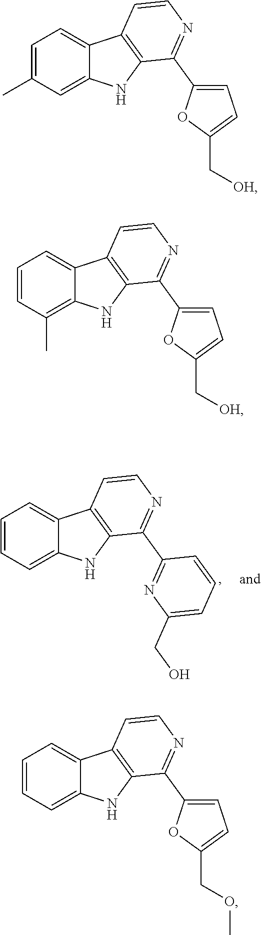

or a pharmaceutically acceptable salt thereof.

In certain embodiments, the disclosure provides a pharmaceutical composition comprising a compound described herein and a pharmaceutically acceptable excipient.

In certain aspects, the present disclosure provides methods of treating or preventing inflammation or treating or preventing a viral infection, comprising administering to a subject in need thereof a therapeutically effective amount of a cell-free extract from a vaginal bacterial strain.

### BRIEF DESCRIPTION OF THE DRAWINGS

FIG. 1 is a graph that shows that compounds described herein are suppressive at higher concentrations.

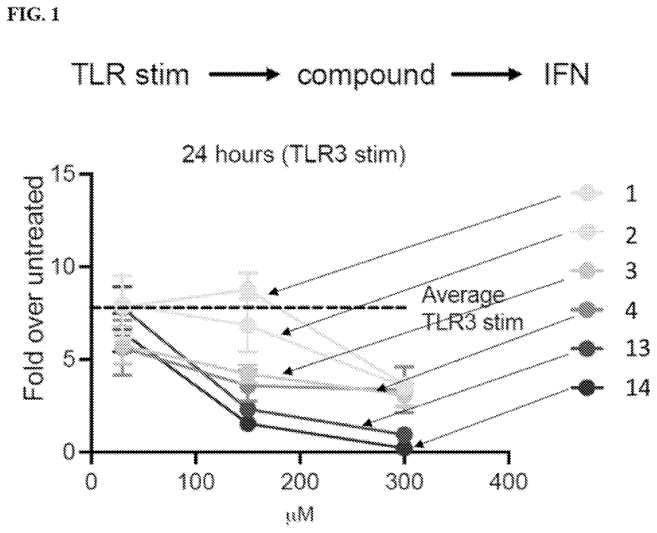

FIG. 2 is a graph that shows that compounds described herein are suppressive at higher concentrations.

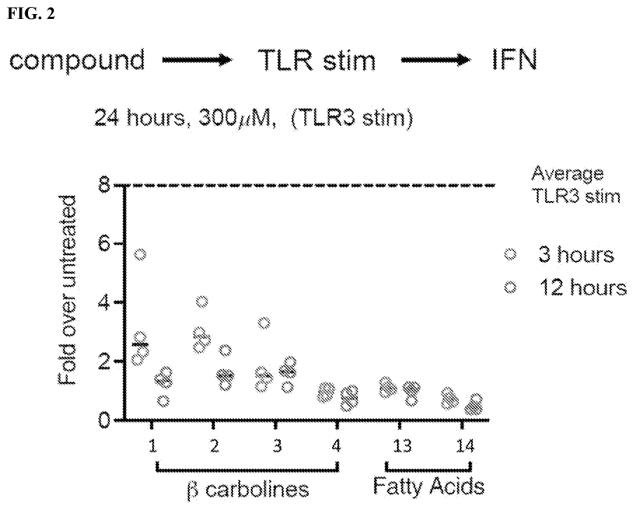

FIG. 3 is a graph that shows that reducing the concentration resulted in a partial hit.

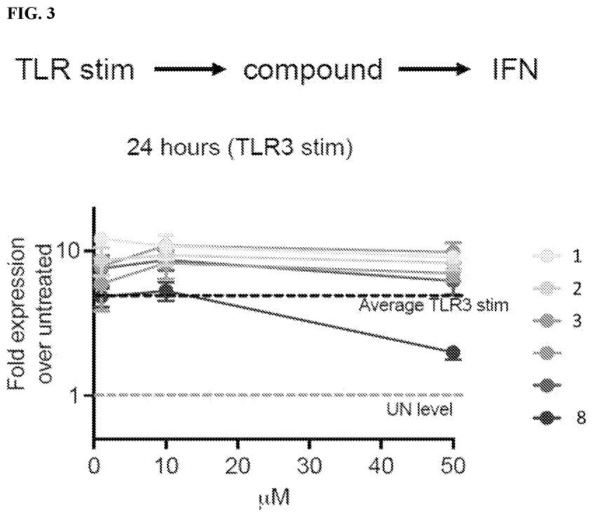

FIG. 4 is a graph that shows that reducing the concentration resulted in a partial hit.

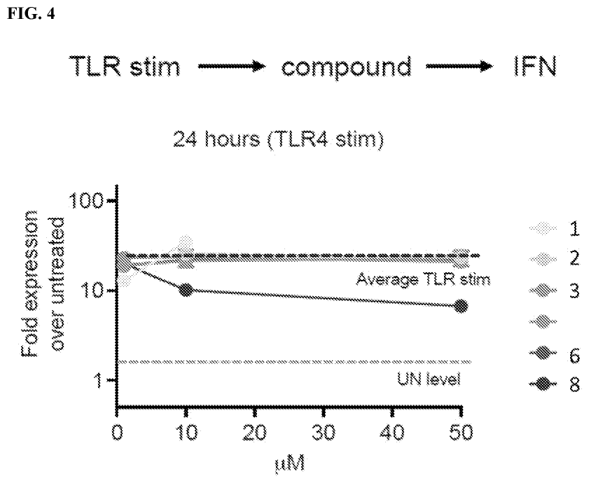

FIG. 5 is a graph that shows that pretreating (3-12 h) keeps TLR4 stim at close to untreated levels (compound→TLR stim→IFN; 24 h, 10 μM, 3 h treatment, TLR4 stim).

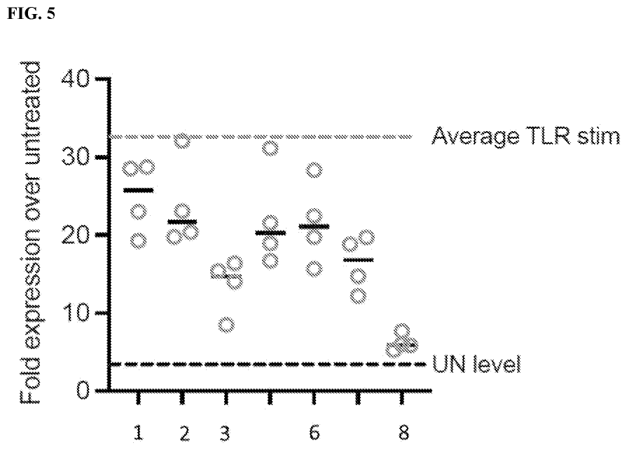

FIG. 6A is a graph showing the suppressive activity of compounds described herein (100 μM) after 1 h pre-treatment.

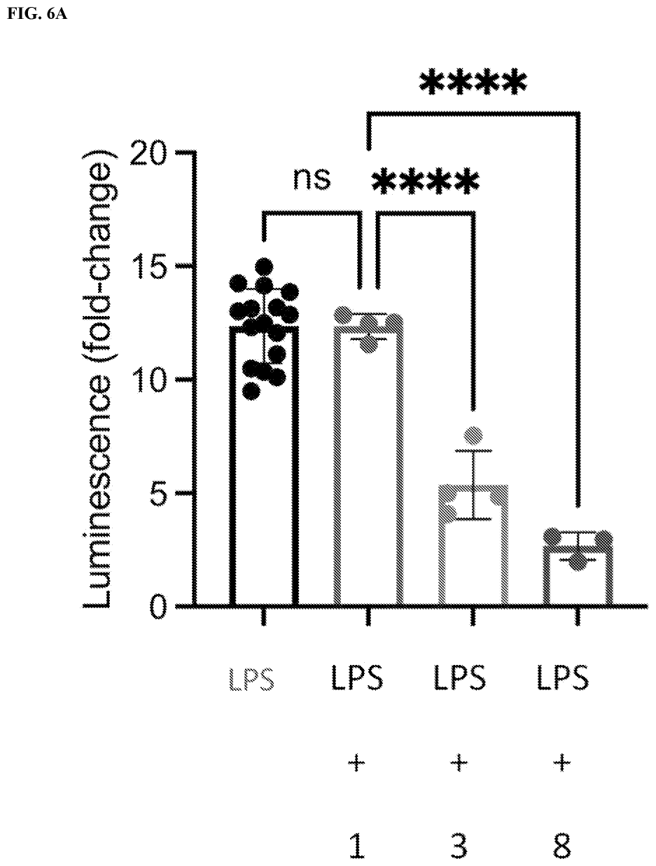

FIG. 6B is a graph showing the suppressive activity of compounds described herein (100 μM) after 1 h post-treatment.

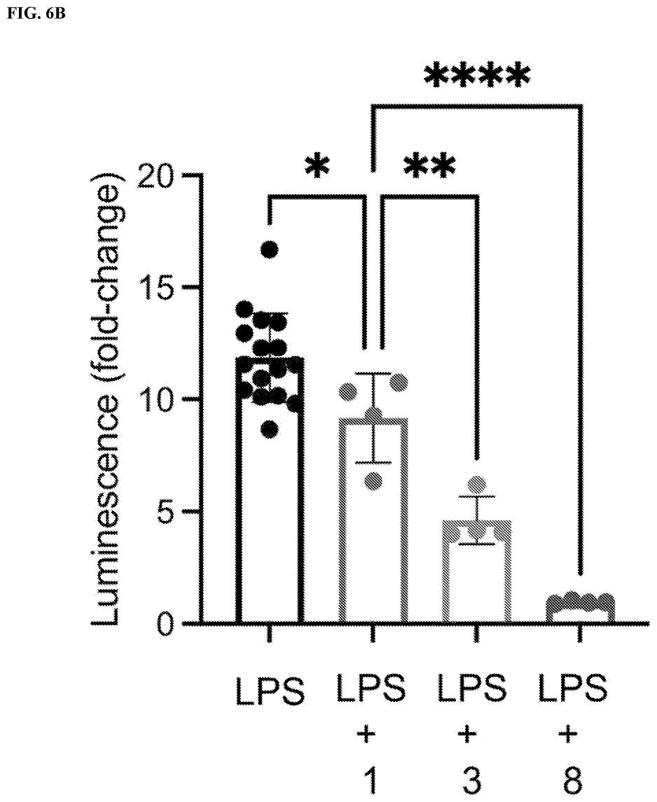

FIG. 7 is a graph showing suppression of NFκB by compounds described herein at various concentrations.

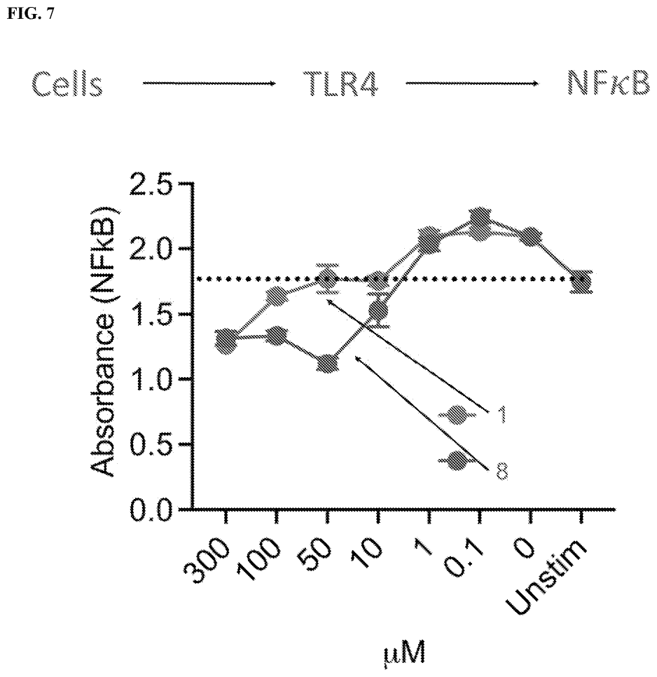

FIG. 8A is a graph showing the suppression of interferon signaling by compounds described herein at various concentrations after 1 h pre-treatment.

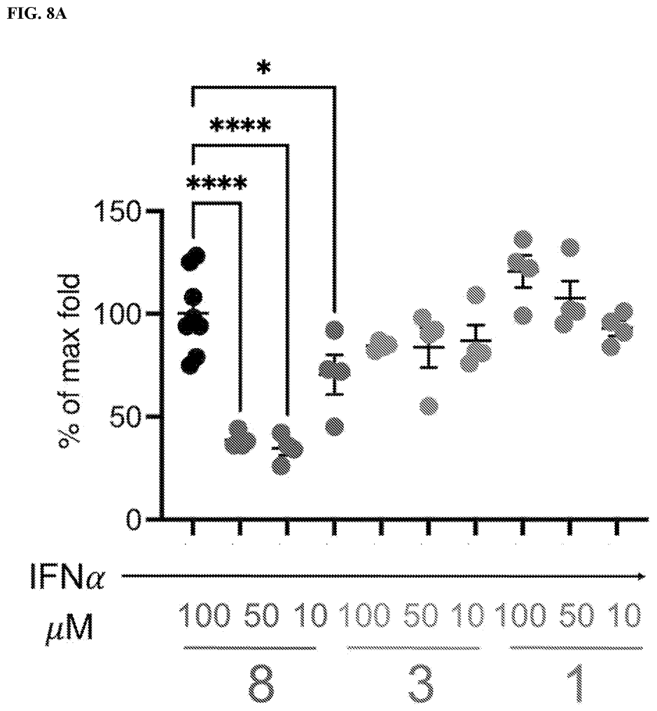

FIG. 8B is a graph showing the suppression of interferon signaling by compounds described herein at various concentrations after 1 h post-treatment.

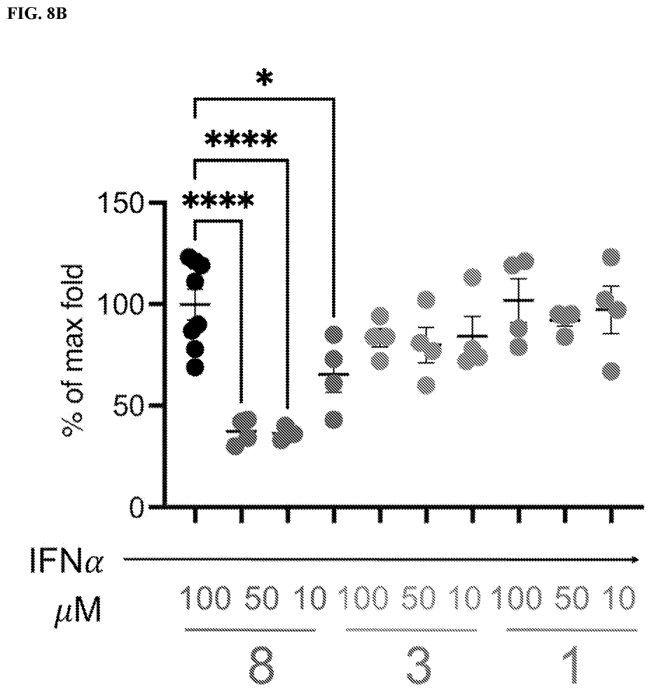

FIG. 9A is a series of graphs showing the suppression of NFκB induction in primary monocytes by compound 8.

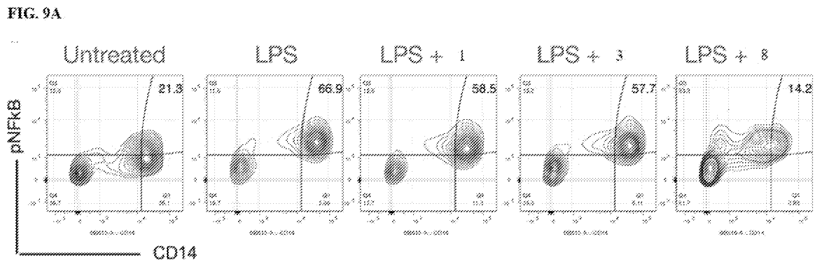

FIG. 9B is a graph showing the suppression of NFκB induction in primary monocytes by compound 8.

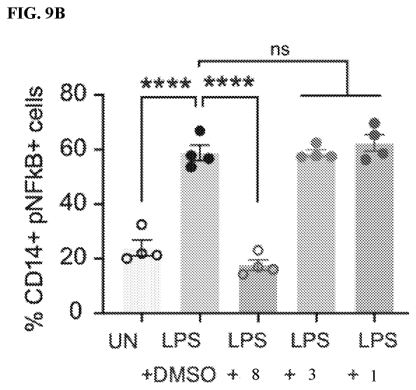

FIG. 9C is a graph showing the suppression of NFκB induction in primary monocytes by compound 8.

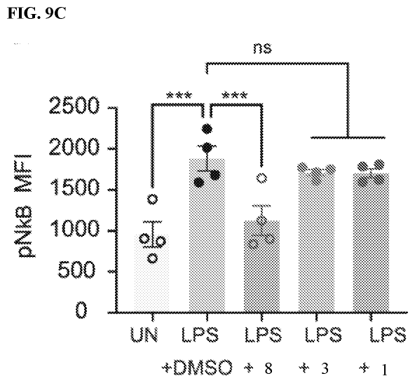

FIG. 10 is a graph showing percentage of maximum TLR4 activity for various compounds described herein at 10 μM after 24 h.

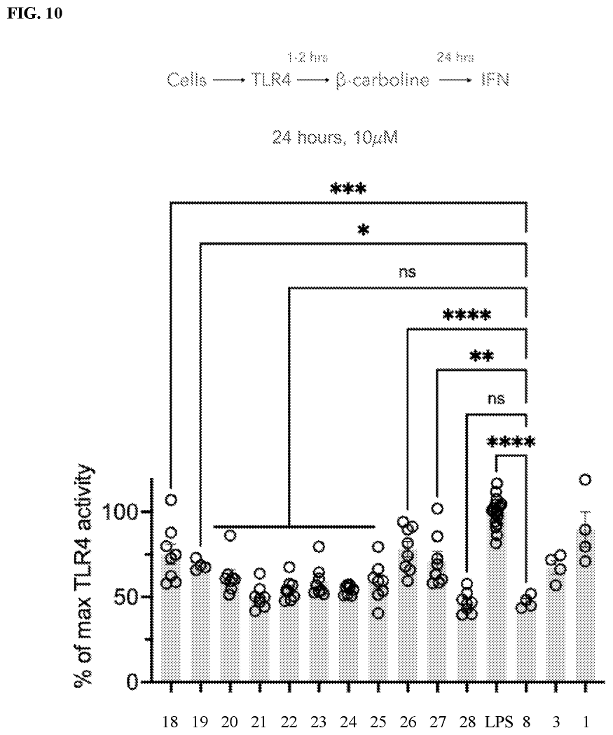

### DETAILED DESCRIPTION

Applicant discovered that targeted activation of the immune cells in the vagina can successfully protect the entire mucosa against infection. As a result of this discovery, described herein are broadly effective topical anti-inflammatory compounds for use in vaginal and other mucosal surfaces. While not wishing to be bound by any particular theory, in some embodiments, the compounds described herein may inhibit recruitment of the inflammatory cell types associated with vaginal inflammation.

In particular embodiments, the invention relates to any of the methods described herein, wherein the compound is selected from Table 1, or a pharmaceutically acceptable salt thereof.

TABLE 1

| Compound No. | Structure |
|---|---|
| 1 | 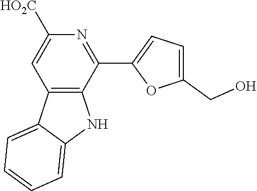 [SMILES: O=C(O)c1cc2c([nH]c3ccccc32)c(-c2ccc(CO)o2)n1] |
| 2 | 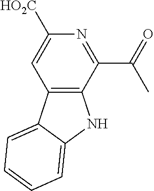 [SMILES: CC(=O)c1nc(C(=O)O)cc2c1[nH]c1ccccc12] |
| 3 | 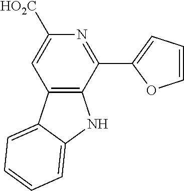 [SMILES: O=C(O)c1cc2c([nH]c3ccccc32)c(-c2ccco2)n1] |
| 4 | 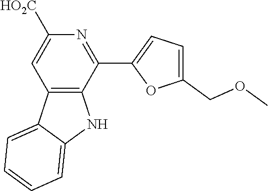 [SMILES: COCc1ccc(-c2nc(C(=O)O)cc3c2[nH]c2ccccc23)o1] |
| 5 | 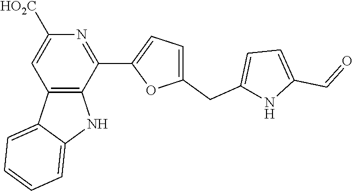 [SMILES: O=Cc1ccc(Cc2ccc(-c3nc(C(=O)O)cc4c3[nH]c3ccccc34)o2)[nH]1] |
| 6 | 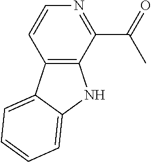 [SMILES: CC(=O)c1nccc2c1[nH]c1ccccc12] |
| 7 | 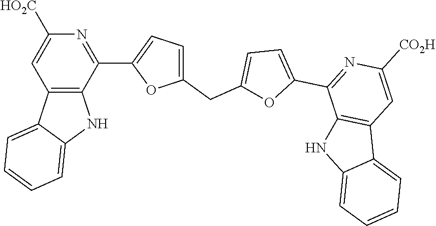 [SMILES: O=C(O)c1cc2c([nH]c3ccccc32)c(-c2ccc(Cc3ccc(-c4nc(C(=O)O)cc5c4[nH]c4ccccc45)o3)o2)n1] |
| 8 | 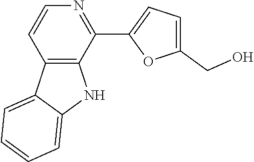 [SMILES: OCc1ccc(-c2nccc3c2[nH]c2ccccc23)o1] |
| 9 | 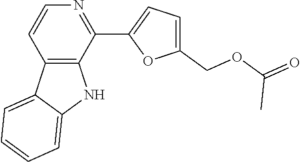 [SMILES: CC(=O)OCc1ccc(-c2nccc3c2[nH]c2ccccc23)o1] |
| 10 | 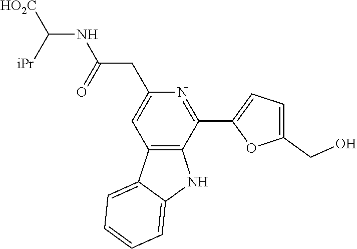 [SMILES: CC(C)C(NC(=O)Cc1cc2c([nH]c3ccccc32)c(-c2ccc(CO)o2)n1)C(=O)O] |
| 11 | 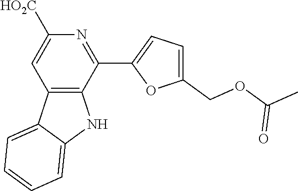 [SMILES: CC(=O)OCc1ccc(-c2nc(C(=O)O)cc3c2[nH]c2ccccc23)o1] |
| 18 | 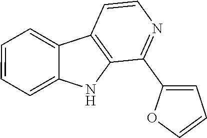 [SMILES: c1coc(-c2nccc3c2[nH]c2ccccc23)c1] |
| 19 | 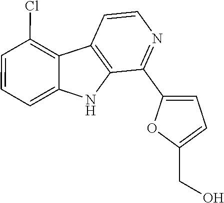 [SMILES: OCc1ccc(-c2nccc3c2[nH]c2cccc(Cl)c23)o1] |
| 20 | 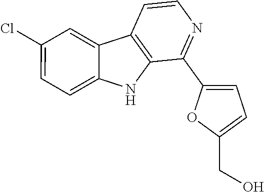 [SMILES: OCc1ccc(-c2nccc3c2[nH]c2ccc(Cl)cc23)o1] |
| 21 | 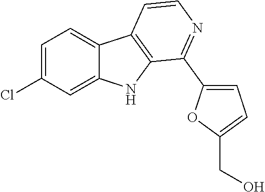 [SMILES: OCc1ccc(-c2nccc3c2[nH]c2cc(Cl)ccc23)o1] |
| 22 | 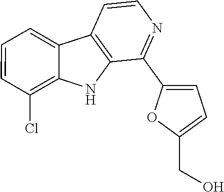 [SMILES: OCc1ccc(-c2nccc3c2[nH]c2c(Cl)cccc23)o1] |
| 23 | 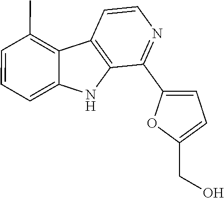 [SMILES: Cc1cccc2[nH]c3c(-c4ccc(CO)o4)nccc3c12] |
| 24 | 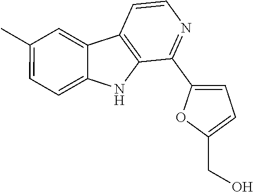 [SMILES: Cc1ccc2[nH]c3c(-c4ccc(CO)o4)nccc3c2c1] |
| 25 | 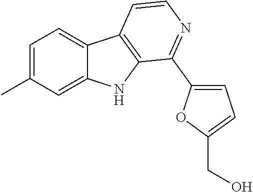 [SMILES: Cc1ccc2c(c1)[nH]c1c(-c3ccc(CO)o3)nccc12] |
| 26 | 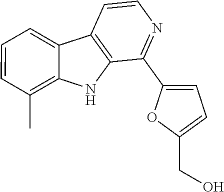 [SMILES: Cc1cccc2c1[nH]c1c(-c3ccc(CO)o3)nccc12] |
| 27 | 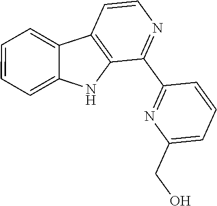 [SMILES: OCc1cccc(-c2nccc3c2[nH]c2ccccc23)n1] |
| 28 | 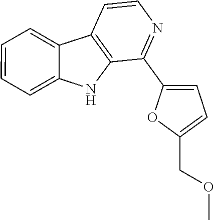 [SMILES: COCc1ccc(-c2nccc3c2[nH]c2ccccc23)o1] |

In particular embodiments, the invention relates to any of the methods described herein, wherein the compound is selected from Table 2, or a pharmaceutically acceptable salt thereof.

TABLE 2

| Compound | |
|---|---|
| No. | Structure |
| 12 | 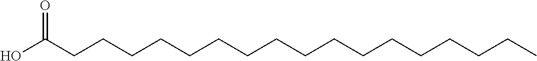 [SMILES: CCCCCCCCCCCCCCCCCC(=O)O] |
| 13 | 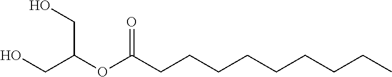 [SMILES: CCCCCCCCCC(=O)OC(CO)CO] |
| 14 | 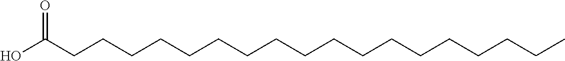 [SMILES: CCCCCCCCCCCCCCCCCCC(=O)O] |
| 15 | 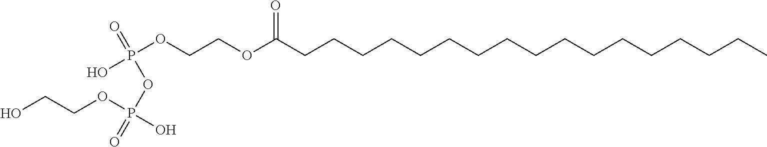 [SMILES: CCCCCCCCCCCCCCCCCC(=O)OCCOP(=O)(O)OP(=O)(O)OCCO] |
| 16 | 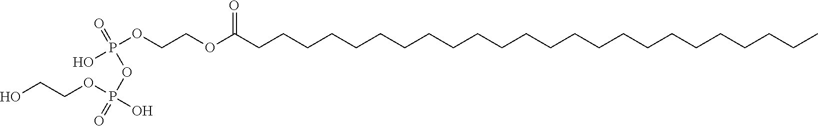 [SMILES: CCCCCCCCCCCCCCCCCCCCCCCCC(=O)OCCOP(=O)(O)OP(=O)(O)OCCO] |
| 17 | 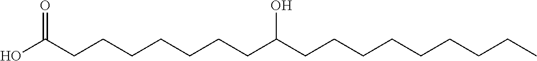 [SMILES: CCCCCCCCCC(O)CCCCCCCC(=O)O] |

### Pharmaceutical Compositions

In certain embodiments, the compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound described herein and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound described herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound described herein. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound described herein, with the carrier and, optionally, one or more accessory ingredients. In general, the formulations are prepared by uniformly and intimately bringing into association a compound with liquid carriers, or finely divided solid carriers, or both, and then, if necessary, shaping the product.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound described herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound described herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound described herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods described herein will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds described herein may be used alone or conjointly administered with another type of therapeutic agent.

The present disclosure includes the use of pharmaceutically acceptable salts of compounds described herein in the compositions and methods described herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn or other metal salts. In certain embodiments, contemplated salts include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, or diminishes the severity or length of (when administered prior to onset of the symptoms), symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered topically, intravaginally, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents).

For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt that is suitable for or compatible with the treatment of patients.

The term “pharmaceutically acceptable acid addition salt” as used herein means any non-toxic organic or inorganic salt of any base compounds. Illustrative inorganic acids that form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds for laboratory use, or for subsequent conversion to a pharmaceutically acceptable acid addition salt.

The term “pharmaceutically acceptable basic addition salt” as used herein means any non-toxic organic or inorganic base addition salt of any acid. Illustrative inorganic bases that form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

As used herein except where noted, “alkyl” is intended to include both branched- and straight-chain saturated aliphatic hydrocarbon groups, including all isomers, having the specified number of carbon atoms. Commonly used abbreviations for alkyl groups are used throughout the specification, e.g. methyl may be represented by “Me” or CH₃, ethyl may be represented by “Et” or CH₂CH₃, propyl may be represented by “Pr” or CH₂CH₂CH₃, butyl may be represented by “Bu” or CH₂CH₂CH₂CH₃, etc. “C₁₋₆ alkyl” (or “C₁-C₆ alkyl”) for example, means linear or branched chain alkyl groups, including all isomers, having the specified number of carbon atoms. C₁₋₆ alkyl includes all of the hexyl alkyl and pentyl alkyl isomers as well as n-, iso-, sec- and t-butyl, n- and isopropyl, ethyl and methyl. “C₁₋₄ alkyl” means n-, iso-, sec- and t-butyl, n- and isopropyl, ethyl and methyl. The term “alkylene” refers to both branched- and straight-chain saturated aliphatic hydrocarbon groups, including all isomers, having the specified number of carbons, and having two terminal end chain attachments. For illustration, the term “unsubstituted A-C₄ alkylene-B” represents A-CH₂—CH₂—CH₂—CH₂—B.

Unless otherwise specifically noted as only “unsubstituted” or only “substituted”, or when substituents are enumerated, alkyl (either as a stand alone radical or as part of a radical such as alkoxy, alkylthio and aralkyl) groups are unsubstituted or substituted with 1 to 3 substituents on each carbon atom, with halo, C₁₋₂₀ alkyl, CF₃, NH₂, N(C₁₋₆ alkyl)₂, NO₂, oxo, CN, N₃, —OH, —O(C₁₋₆ alkyl), C₃₋₁₀ cycloalkyl, C₂₋₆ alkenyl, C₂₋₆ alkynyl, (C₀₋₆ alkyl)C(O)NH—, (C₀₋₆ alkyl)C(O)—, (C₀₋₆ alkyl)OC(O)—, (C₀₋₆ alkyl)O(C₁₋₆ alkyl)-, aryl, aralkyl, heterocycle, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl.

The term “C₀” as employed in expressions such as “C₀₋₆ alkyl” means a direct covalent bond; or when the term appears at the terminus of a substituent, C₀₋₆ alkyl means hydrogen or C₁₋₆alkyl. Similarly, when an integer defining the presence of a certain number of atoms in a group is equal to zero, it means that the atoms adjacent thereto are connected directly by a bond.

### EMBODIMENTS

In certain embodiments, the disclosure relates to a method of treating or preventing inflammation or treating or preventing a viral infection, comprising administering to a subject in need thereof a therapeutically effective amount of a compound of formula (Ia):

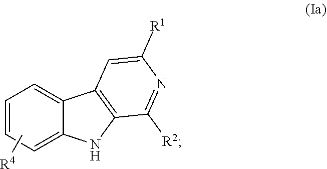

or a pharmaceutically acceptable salt thereof,
wherein
   R¹ is selected from H,

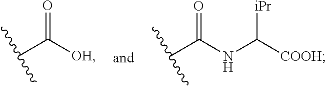

R² is selected from

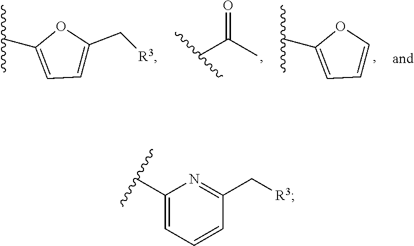

R³ is selected from OH, OCH₃,

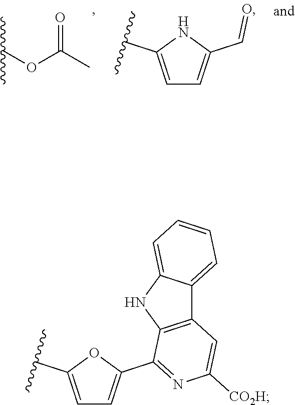

and
R⁴ is selected from H, halo, and C₁-C₄ alkyl.

In certain embodiments, the disclosure relates to any of the methods described herein, wherein the compound of formula (Ia) is a compound of formula (I):

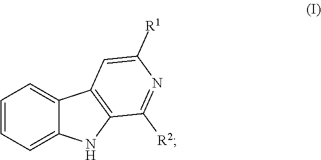

or a pharmaceutically acceptable salt thereof,
wherein
   R¹ is selected from H,

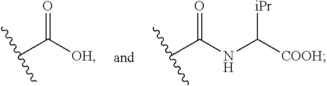

R² is selected from

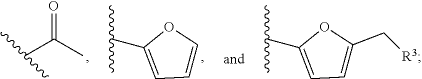

and
R³ is selected from OH, OCH₃,

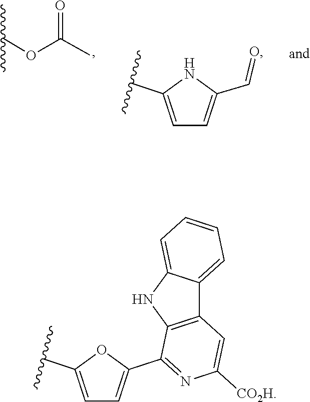

In certain embodiments, the disclosure relates to any of the methods described herein, wherein R¹ is

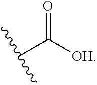 [SMILES: *C(=O)O]

In certain embodiments, the disclosure relates to any of the methods described herein, wherein R¹ is H.

In certain embodiments, the disclosure relates to any of the methods described herein, wherein R² is

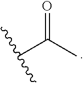 [SMILES: *C(C)=O]

In certain embodiments, the disclosure relates to any of the methods described herein, wherein R² is

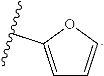 [SMILES: *c1ccco1]

In certain embodiments, the disclosure relates to any of the methods described herein, wherein R² is

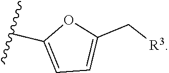

In certain embodiments, the disclosure relates to any of the methods described herein, wherein R³ is OH or

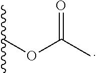 [SMILES: *OC(C)=O]

In certain embodiments, the disclosure relates to any of the methods described herein, wherein R³ is OCH₃.

In certain embodiments, the disclosure relates to any of the methods described herein, wherein the compound is selected from:

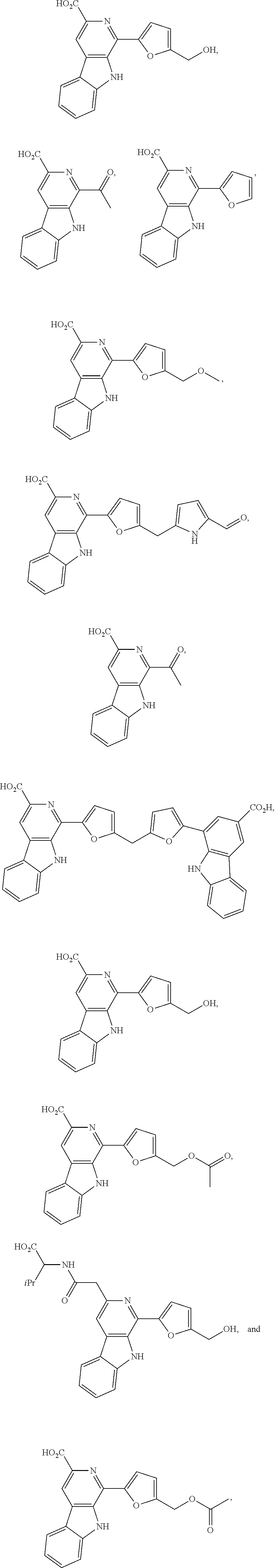

or a pharmaceutically acceptable salt thereof.

In certain embodiments, the disclosure relates to any of the methods described herein, wherein the compound is selected from

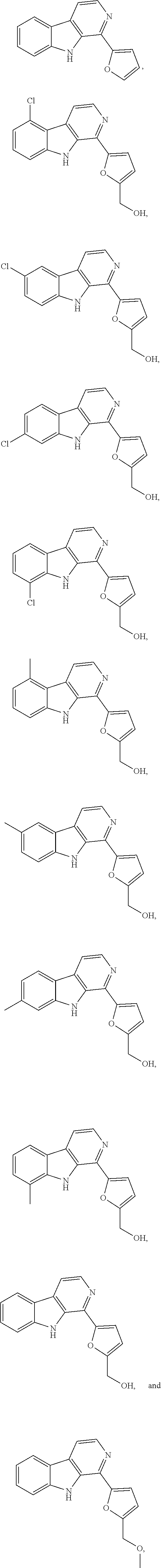

or a pharmaceutically acceptable salt thereof.

In certain embodiments, the disclosure relates to a method of treating or preventing inflammation or treating or preventing a viral infection, comprising administering to a subject in need thereof a therapeutically effective amount of a compound of formula (II):

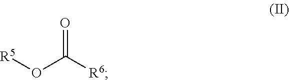

or a pharmaceutically acceptable salt thereof,
wherein
   R⁵ is selected from H,

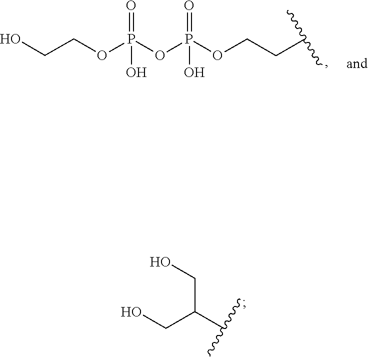 [SMILES: *CCOP(=O)(O)OP(=O)(O)OCCO ; *C(CO)CO]

and
R⁶ is optionally substituted C₈-C₂₆ alkyl.

In certain embodiments, the disclosure relates to any of the methods described herein, wherein R⁶ is substituted with OH. In certain embodiments, the disclosure relates to any of the methods described herein, wherein R⁶ is selected from C₉ alkyl, C₁₇ alkyl, C₁₈ alkyl, and C₂₄ alkyl. In certain embodiments, the disclosure relates to any of the methods described herein, wherein R⁶ is C₉ alkyl. In certain embodiments, the disclosure relates to any of the methods described herein, wherein R⁶ is C₁₇ alkyl. In certain embodiments, the disclosure relates to any of the methods described herein, wherein R⁶ is C₁₈ alkyl. In certain embodiments, the disclosure relates to any of the methods described herein, wherein R⁶ is C₂₄ alkyl.

In certain embodiments, the disclosure relates to any of the methods described herein, wherein R⁵ is H. In certain embodiments, the disclosure relates to any of the methods described herein, wherein R⁵ is

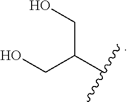 [SMILES: *C(CO)CO]

In certain embodiments, the disclosure relates to any of the methods described herein, wherein R⁵ is

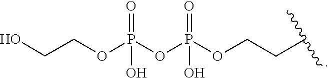 [SMILES: *CCOP(=O)(O)OP(=O)(O)OCCO]

In certain embodiments, the disclosure relates to any of the methods described herein, wherein the compound is selected from:

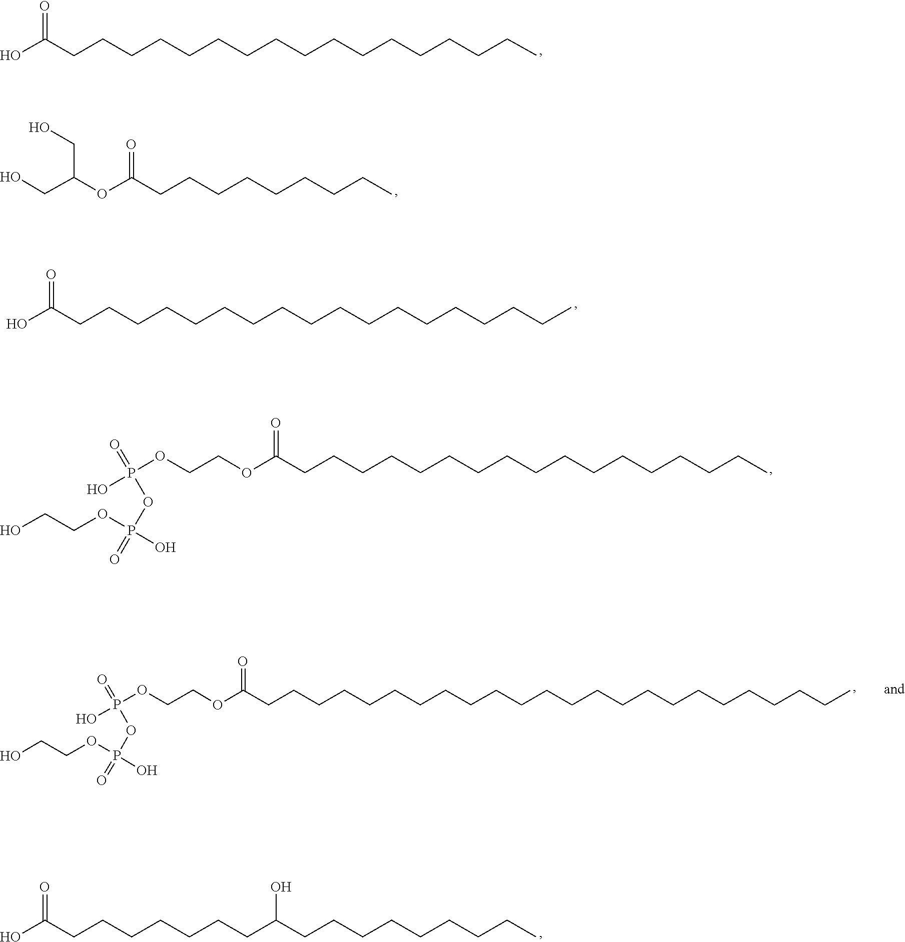 [SMILES: CCCCCCCCCCCCCCCCCC(=O)O ; CCCCCCCCCC(=O)OC(CO)CO ; CCCCCCCCCCCCCCCCCCC(=O)O ; CCCCCCCCCCCCCCCCCC(=O)OCCOP(=O)(O)OP(=O)(O)OCCO ; CCCCCCCCCCCCCCCCCCCCCCCCC(=O)OCCOP(=O)(O)OP(=O)(O)OCCO ; CCCCCCCCCC(O)CCCCCCCC(=O)O]

or a pharmaceutically acceptable salt thereof.

In certain embodiments, the disclosure relates to any of the methods described herein, wherein the compound is administered topically.

In certain embodiments, the disclosure relates to any of the methods described herein, wherein the compound is administered intravaginally.

In certain embodiments, the disclosure relates to any of the methods described herein, the compound is administered to a mucosal surface of the subject. In certain embodiments, the disclosure relates to any of the methods described herein, wherein the mucosal surface is in the subject's vagina.

In certain embodiments, the disclosure relates to any of the methods described herein, wherein the method is a method of treating inflammation. In certain embodiments, the disclosure relates to any of the methods described herein, wherein the method is a method of treating bacterial vaginosis. In certain embodiments, the disclosure relates to any of the methods described herein, wherein the method is a method of treating vaginitis.

In certain embodiments, the disclosure relates to any of the methods described herein, wherein the subject is suffering from pruritus.

In certain embodiments, the disclosure relates to any of the methods described herein, wherein the subject is suffering from pain.

In certain embodiments, the disclosure relates to any of the methods described herein, wherein the method is a method of preventing inflammation.

In certain embodiments, the disclosure relates to any of the methods described herein, wherein the method is a method of treating a viral infection.

In certain embodiments, the disclosure relates to any of the methods described herein, wherein the method is a method of preventing a viral infection.

In certain embodiments, the disclosure relates to any of the methods described herein, wherein the method is a method of preventing genital herpes.

In certain embodiments, the disclosure relates a compound selected from:

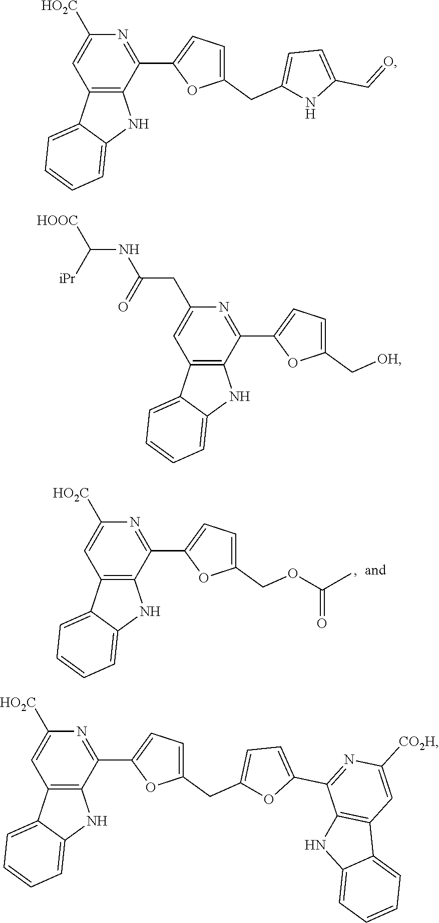

pharmaceutically acceptable salt thereof.

In certain embodiments, the disclosure relates to a compound selected from

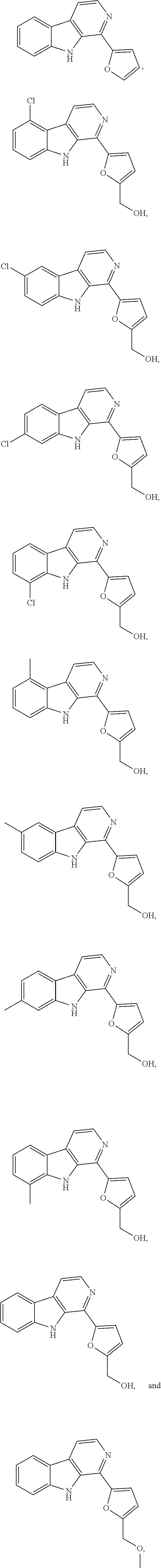

or a pharmaceutically acceptable salt thereof.

In certain embodiments, the disclosure relates to a pharmaceutical composition comprising any of the compounds described herein and a pharmaceutically acceptable excipient.

### EXAMPLES

The invention now being generally described, it will be more readily understood by reference to the following examples, which are included merely for purposes of illustration of certain aspects and embodiments of the present invention, and are not intended to limit the invention.

### Example 1

Filtered bacterial supernatant from over 30 vaginal bacterial strains were collected, with intestinal and mouse lactobacilli strains as controls. This supernatant was added to human macrophages previously activated by Toll Like Receptor (TLR) ligands, which activate secretion of pro-inflammatory cytokines. Reporter cells showing activation of transcription factors NFκB and interferon, which together control induction of inflammatory cytokines associated with vaginal inflammation such as IL-1α, IL-8 and TNF-α, were used. Addition of filtered supernatant from vaginal lactobacilli, but not intestinal lactobacilli, suppress activation of human macrophages by LPS, a TLR4 ligand (data not shown). These data represent a candidate strain from each species. At least 4 and up to 8 strains of each vaginal lactobacilli species were tested subsequently. Similar suppression of TLR2 and TLR3 signaling pathways were also observed. Active fractions from the supernatant of a candidate vaginal lactobacilli strain were identified (data not shown).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing inflammation or treating or preventing a viral infection, comprising administering to a subject in need thereof a therapeutically effective amount of a compound of formula (Ia): 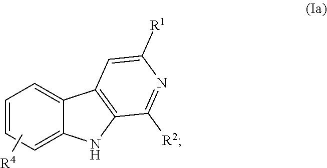
or a pharmaceutically acceptable salt thereof,
wherein
R¹ is selected from H, 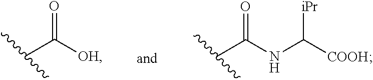
R² is selected from 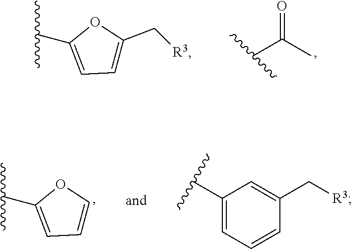
R³ is selected from OH, OCH₃, 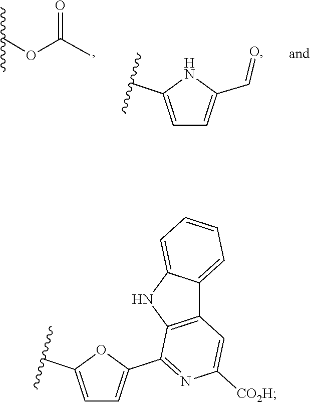
and
R⁴ is selected from H, halo, and C₁-C₄ alkyl.

2. The method of claim 1, wherein the compound of formula (Ia) is a compound of formula (I): 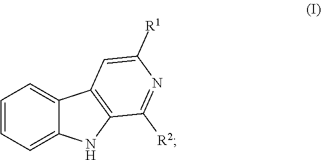
or a pharmaceutically acceptable salt thereof,
wherein
R¹ is selected from H, 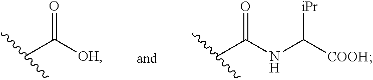
R² is selected from 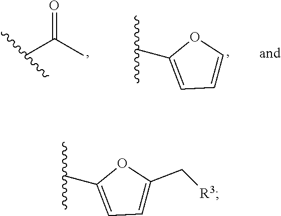
and
R³ is selected from OH, OCH₃,

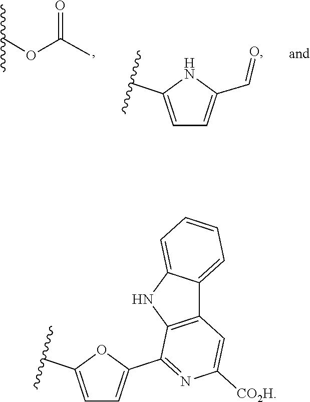

3. The method of claim 1, wherein R¹ is 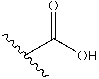 [SMILES: *C(=O)O]
or H.

4. (canceled)

5. The method of claim 1, wherein R² is

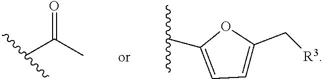

6.-7. (canceled)

8. The method of claim 1, wherein R³ is selected from OCH₃, OH, and

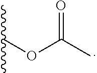 [SMILES: *OC(C)=O]

9. (canceled)

10. The method of claim 1, wherein the compound is selected from: 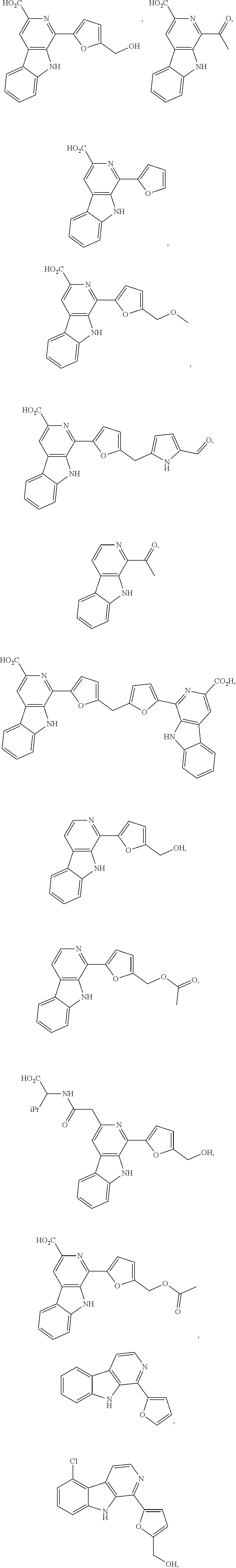 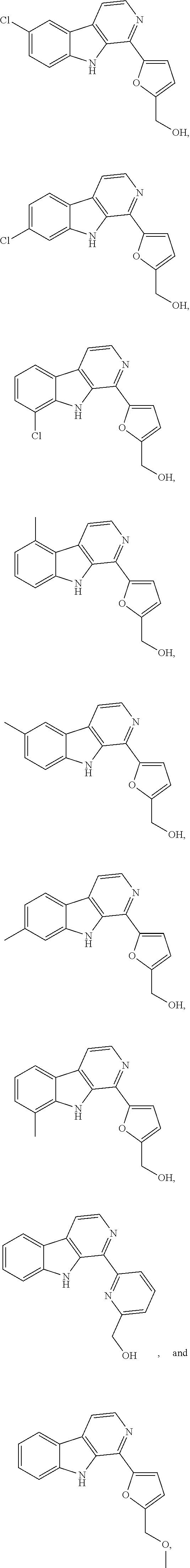
or a pharmaceutically acceptable salt thereof.

11. (canceled)

12. A method of treating or preventing inflammation or treating or preventing a viral infection, comprising administering to a subject in need thereof a therapeutically effective amount of a compound of formula (II): 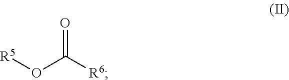
or a pharmaceutically acceptable salt thereof,
wherein
R⁵ is selected from H, 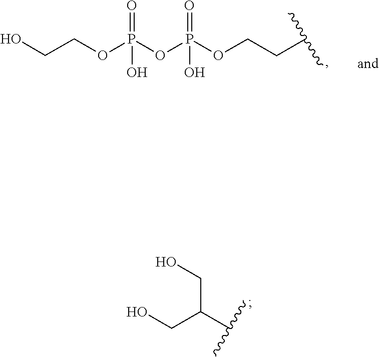 [SMILES: *CCOP(=O)(O)OP(=O)(O)OCCO ; *C(CO)CO]
and
R⁶ is optionally substituted C₈-C₂₆ alkyl.

13. The method of claim 12, wherein R⁶ is substituted with OH.

14. The method of claim 12, wherein R⁶ is selected from C₉ alkyl, C₁₇ alkyl, C₁₈ alkyl, and C₂₄ alkyl.

15.-18. (canceled)

19. The method of claim 12, wherein R⁵ is H or

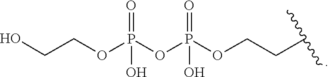 [SMILES: *CCOP(=O)(O)OP(=O)(O)OCCO]

20.-21. (canceled)

22. The method of claim 12, wherein the compound is selected from: 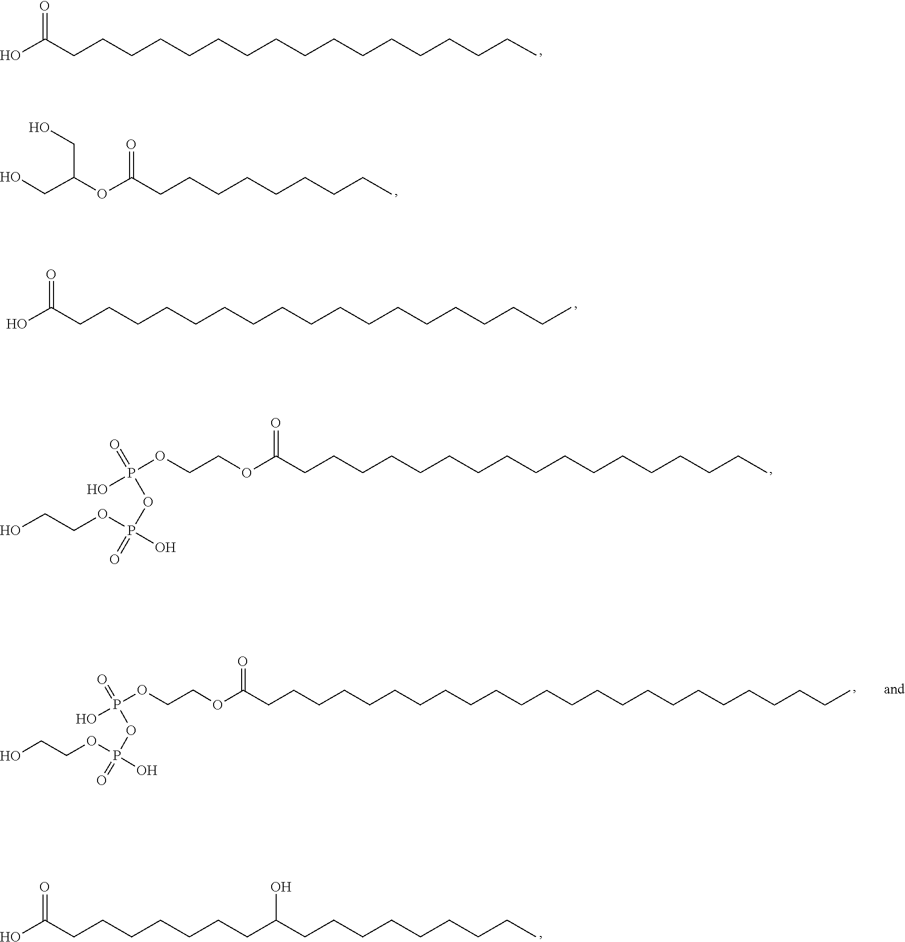 [SMILES: CCCCCCCCCCCCCCCCCC(=O)O ; CCCCCCCCCC(=O)OC(CO)CO ; CCCCCCCCCCCCCCCCCCC(=O)O ; CCCCCCCCCCCCCCCCCC(=O)OCCOP(=O)(O)OP(=O)(O)OCCO ; CCCCCCCCCCCCCCCCCCCCCCCCC(=O)OCCOP(=O)(O)OP(=O)(O)OCCO ; CCCCCCCCCC(O)CCCCCCCC(=O)O]
or a pharmaceutically acceptable salt thereof.

23. The method of claim 1, wherein the compound is administered topically.

24. The method of claim 1, wherein the compound is administered intravaginally.

25.-26. (canceled)

27. The method of claim 1, wherein the method is a method of treating inflammation.

28. The method of claim 27, wherein the method is a method of treating bacterial vaginosis.

29.-32. (canceled)

33. The method of claim 1, wherein the method is a method of treating a viral infection.

34. The method of claim 1, wherein the method is a method of preventing a viral infection.

35. The method of claim 34, wherein the method is a method of preventing genital herpes.

36. A compound selected from: 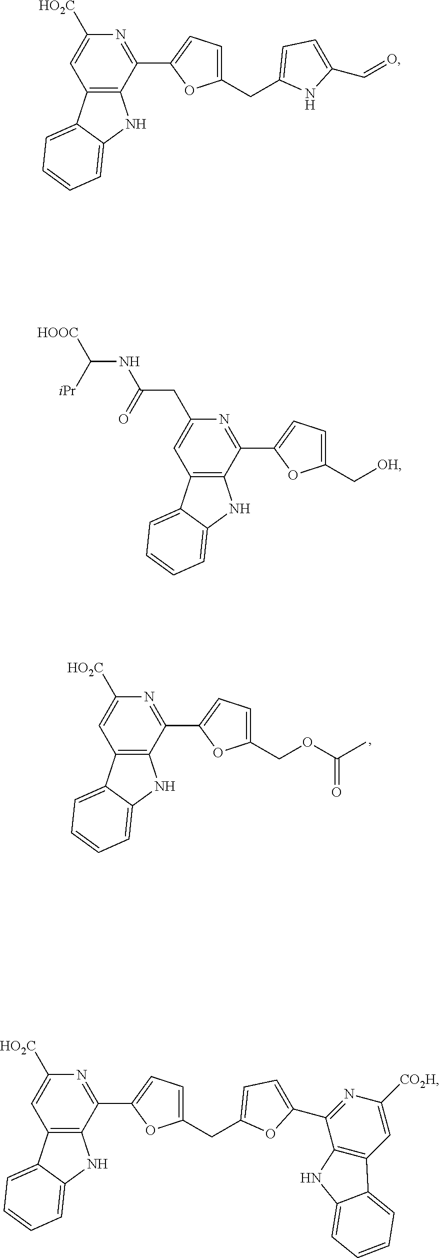 [SMILES: O=Cc1ccc(Cc2ccc(-c3nc(C(=O)O)cc4c3[nH]c3ccccc34)o2)[nH]1 ; CC(C)C(NC(=O)Cc1cc2c([nH]c3ccccc32)c(-c2ccc(CO)o2)n1)C(=O)O ; CC(=O)OCc1ccc(-c2nc(C(=O)O)cc3c2[nH]c2ccccc23)o1 ; O=C(O)c1cc2c([nH]c3ccccc32)c(-c2ccc(Cc3ccc(-c4nc(C(=O)O)cc5c4[nH]c4ccccc45)o3)o2)n1] 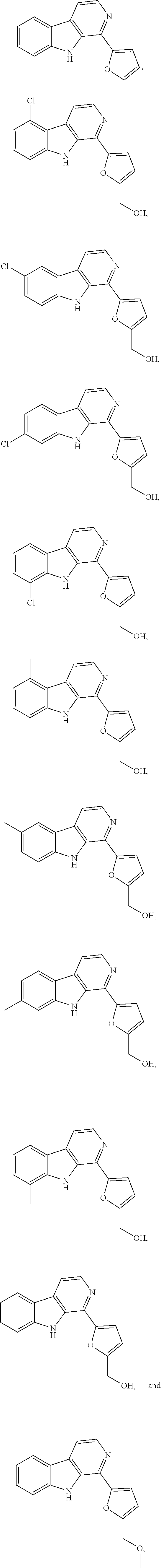
or a pharmaceutically acceptable salt thereof.

37. (canceled)

38. A pharmaceutical composition comprising a compound of claim 36 and a pharmaceutically acceptable excipient.
